(19) United States
(12) Patent Application Publication
(10) Pub. No.: US 2004/0071646 A1
(43) Pub. Date: Apr. 15, 2004

(54) Aerosol device containing a polyurethane and/or polyurea and a fixing polymer

(76) Inventors: Francoise Pataut, Paris (FR); Jean-Luc Bremenson, Paris (FR); Cecile Nocerino, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/451,414

(22) PCT Filed: Dec. 14, 2001
(86) PCT No.: PCT/FR01/04001

(30) Foreign Application Priority Data
Dec. 22, 2000 (FR) 00/16950

(51) Int. Cl. A61K 7/06

(52) U.S. Cl. 424/70.11

(57) ABSTRACT

The invention concerns aerosol devices comprising a container which contains, in a cosmetically acceptable medium, a multiple block polymer including at least a polyurethane and/or polyurea unit and at least a fixing polymer whereof the glass transition temperature is not less than 40° C., said devices being suitable for obtaining an initial aerosol flow rate not less than 0.55 grams per second. The invention also concerns a method for hair styling or hair setting comprising the use of said devices and their use for making an aerosol lacquer or spray.

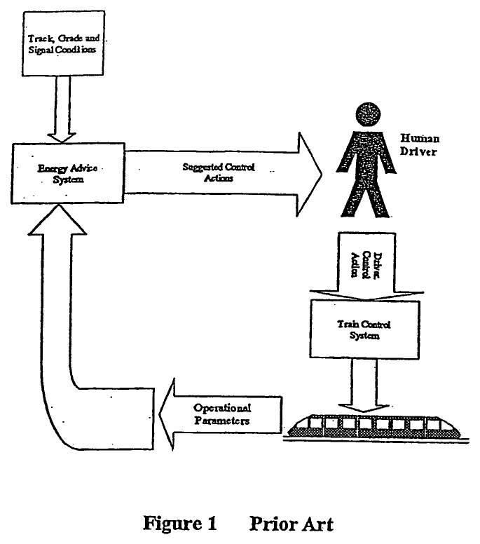

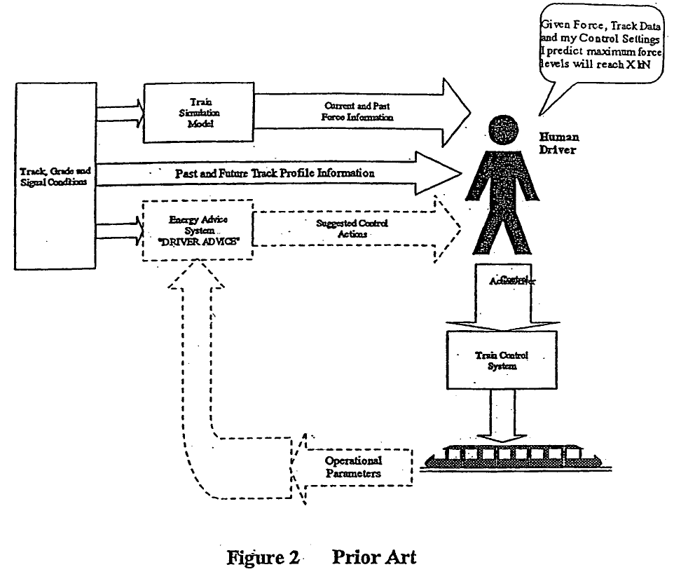

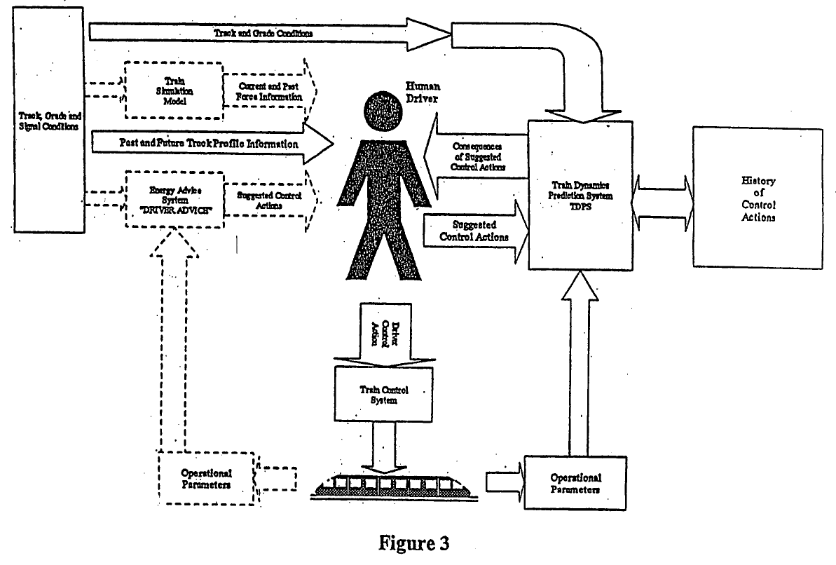

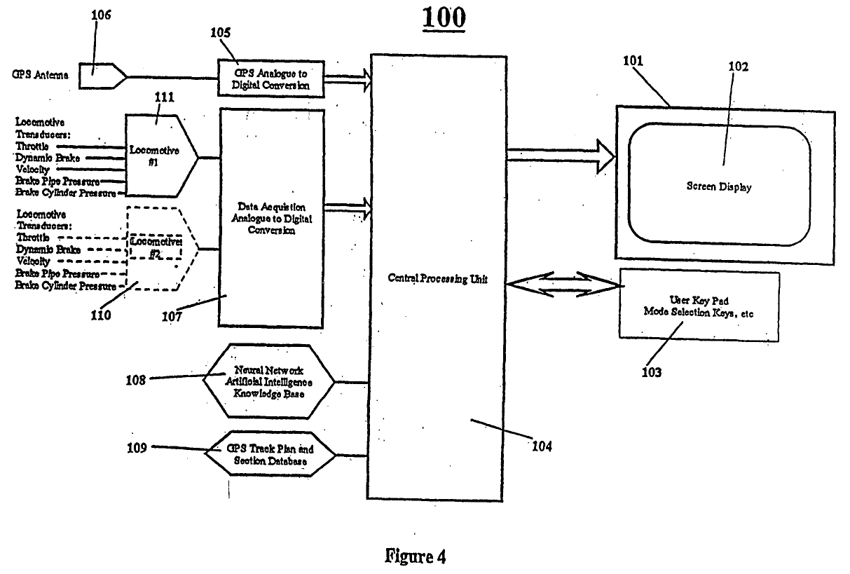

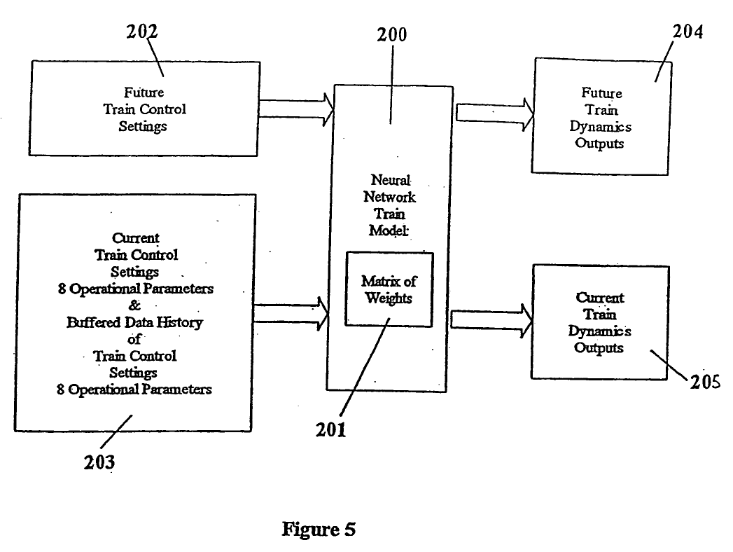

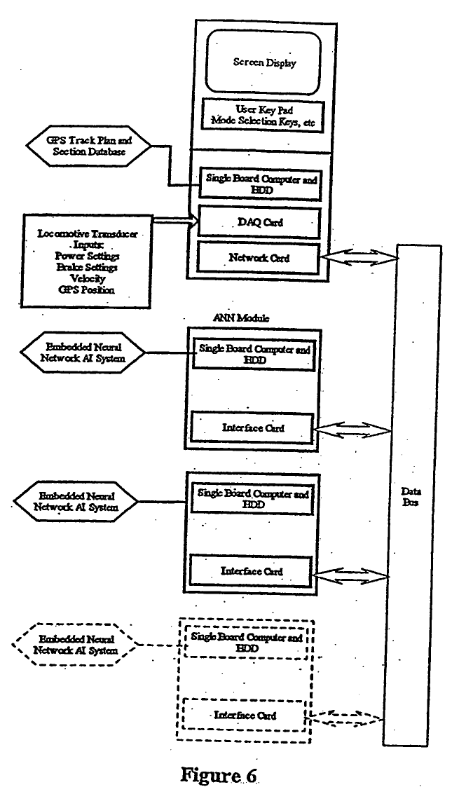

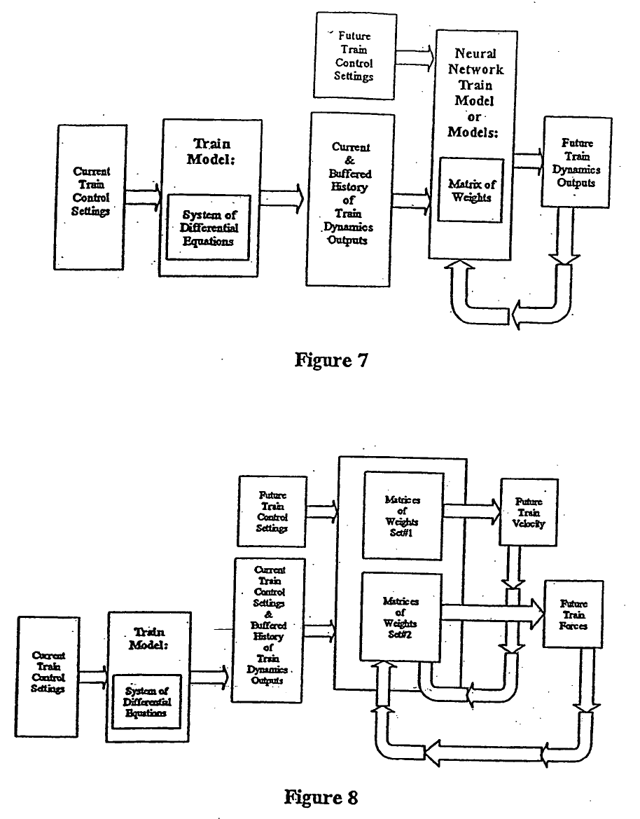

The invention relates to aerosol devices comprising a container that contains, in a cosmetically acceptable medium, a multiblock polymer comprising at least one polyurethane and/or polyurea unit and at least one fixing polymer whose glass transition temperature is greater than or equal to 40° C., these devices being suitable for obtaining an initial flow rate of aerosol composition of greater than or equal to 0.55 gram per second. The invention is also directed toward a process for shaping or holding the hairstyle, comprising the use of these devices, and also to their use for manufacturing an aerosol lacquer or spray.

Fixing the hairstyle is an important part of hair styling, which consists in holding the shape already produced or shaping the hair and simultaneously fixing it. In this field, long-lasting and reliable fixing of the hairstyle is desired, while avoiding rigidly setting the hairstyle.

The hair products for shaping and/or holding the hairstyle that are the most common on the cosmetics market are spray compositions consisting essentially of a solution, usually an alcoholic or aqueous solution, and of one or more materials, generally polymer resins, the function of which is to form welds between the hair, which are also known as fixing materials, as a mixture with various cosmetic adjuvants. This solution may be packaged, for example, in a suitable aerosol container pressurized using a propellant.

Packaging in aerosol form is especially practical for the user, who obtains a relatively uniform distribution of the product without difficulty. However, this type of packaging has the drawback of giving rise to an emission of volatile organic compounds (VOC) that are harmful to the environment. They arise especially from the amount of organic solvent used and from the propellent gas chosen to manufacture the composition. Most particular interest is thus shown in producing cosmetic compositions packaged in aerosol form, for which the amount of volatile organic compounds discharged is lower.

It is known practice from patent DE 198 07 908 to prepare hairstyling compositions comprising a polymer containing a polyurethane unit as fixing polymer. The devices may, however, be improved as especially regards the cosmetic properties they give the hair, while at the same time offering a very good quality of fixing. These devices may also be improved as regards the disentangling of the hair after application of the compositions they contain.

Surprisingly and unexpectedly, the Applicant has discovered, against all expectation, that it is possible to produce aerosol devices that satisfy the requirements stated above, by making a selection firstly of the cosmetic composition, and secondly of the means for distributing this composition.

A subject of the invention is an aerosol device comprising a container containing a hair composition formed from a fluid and at least one propellant, and also means for dispensing the composition, characterized in that:

(1) the composition comprises, in a cosmetically acceptable medium:

(i) between 4.5% and 20% by weight, relative to the total weight of the composition, of at least one polycondensate comprising at least one polyurethane and/or polyurea block;

(ii) between 0.1% and 5% by weight, relative to the total weight of the composition, of at least one fixing polymer other than the polycondensate(s) (i), with a glass transition temperature of between 40 and 200° C.;

(2) the device is suitable for supplying an initial flow rate of aerosol composition of greater than or equal to 0.55 gram per second and less than or equal to 2 grams per second.

Another subject of the invention concerns a process for shaping or holding the hairstyle, comprising the use of this aerosol device.

Yet another subject of the invention concerns the use of this device for the manufacture of an aerosol lacquer or spray.

In accordance with the present invention, the glass transition temperature is measured by DSC (Differential Scanning Calorimetry). According to the present invention, the term “glass transition temperature (Tg)” means the Tg of the fixing material in the dry extract, the dry extract consisting of all of the non-volatile materials in the fluid, or the solids. The glass transition temperature is measured in accordance with the protocol described in patent applications EP 845 257 and EP 848 941, filed by the Applicant, at a temperature of 25° C. ±1° C. and at a relative humidity of 46% ±2%.

The polycondensates comprising at least one polyurethane and/or polyurea block that are particularly targeted by the present invention are those described in patents EP 0 751 162, EP 0 637 600, FR 2 743 297, EP 0 648 485 and EP 814 764, of which the Applicant is the proprietor, and also patents EP 0 656 021 or WO 94/03510 from the company BASF and EP 0 619 111 from the company National Starch.

The polycondensates used in accordance with the invention may be soluble in the cosmetically acceptable medium, especially after neutralization with an organic or mineral base, or alternatively they may form a dispersion in this medium. The dispersion may then comprise at least 0.05% of surfactant allowing the polycondensate to be dispersed and maintained in dispersion.

According to the invention, any type of surfactant may be used in said dispersion, but preferably a nonionic surfactant. The mean particle size of the polycondensate in the dispersion is preferably between 0.1 and 1 micron.

For example, the polycondensate may be formed by an arrangement of blocks, this arrangement being obtained especially from:

(1) at least one compound containing two or more than two active hydrogen atoms per molecule;

(2) at least one diol or a mixture of diols containing acid radicals or salts thereof;

(3) at least one di- or polyisocyanate.

Advantageously, the compounds (1) are chosen from the group comprising diols, diamines, polyesterols and polyetherols, or a mixture thereof.

The compounds (1) that are preferred are linear polyethylene and polypropylene glycols, in particular those obtained by reacting ethylene oxide or propylene oxide with water or diethylene or dipropylene glycol in the presence of sodium hydroxide as catalyst. These polyglycols generally have a molecular weight of between 600 and 20 000.

Other organic compounds that are preferred are those containing mercapto, amino, carboxyl or hydroxyl groups. Among these, mention is made more particularly of polyhydroxy compounds such as polyether diols, polyester diols, polyacetal diols, polyamide diols, polyester polyamide diols, poly(alkylene ether) diols, polythioether diols and polycarbonate diols.

The preferred polyether diols are, for example, the products of condensation of ethylene oxide, of propylene oxide or of tetrahydrofuran, the grafted or block copolymerization or condensation products thereof, such as mixtures of ethylene oxide and propylene oxide condensates, and the products of high-pressure polymerization of olefins with alkylene oxide condensates. Suitable polyethers are prepared, for example, by condensation of alkylene oxides and of polyhydric alcohols, such as ethylene glycol, 1,2-propylene glycol and 1,4-butanediol.

The polyester diols, polyester amides and polyamide diols are preferably saturated and are obtained, for example, from the reaction of saturated or unsaturated polycarboxylic acids with polyhydric alcohols, diamines or polyamines. Adipic acid, succinic acid, phthalic acid, terephthalic acid and maleic acid may be used, for example, to prepare these compounds. Polyhydric alcohols that are suitable for preparing the polyesters include, for example, ethylene glycol, 1,2-propylene glycol, 1,4-butanediol, neopentyl glycol and hexanediol. Amino alcohols may also be used, for example ethanolamine. Diamines that are suitable for preparing the polyester amides are ethylenediamine and hexamethylenediamine.

Suitable polyacetals may be prepared, for example, from 1,4-butanediol or hexanediol and from formaldehyde. Suitable polythioethers may be prepared, for example, by condensation reaction between thioglycols either alone or in combination with other glycols such as ethylene glycol, 1,2-propylene glycol or with other polyhydroxylated compounds. Polyhydroxylated compounds already containing urea or urethane groups, natural polyols, which may be further modified, for example castor oil and carbohydrates, may also be used.

More preferably, the compound of group (1) is a polyesterol, especially a polyester diol formed by the reaction of at least one (di) -polyol (1ₐ) and of at least one acid (1_{b}). The (di) -polyol (1ₐ) is chosen in particular from the group comprising neopentyl glycol, 1,4-butanediol, hexanediol, ethylene glycol, diethylene glycol, propylene glycol, butylene glycol, neopentyl glycol and (di)polyethylene glycol. The acid (1_{b}) is chosen in particular from the group comprising phthalic acid, isophthalic acid, adipic acid and (poly)lactic acid.

Compounds (2) that may especially be used include a hydroxycarboxylic acid such as dimethylolpropanoic acid (DMPA) or a 2,2-hydroxymethylcarboxylic acid. In general, compound (2) is useful as coupling block. Preferred compounds (2) include those comprising at least one poly((α-hydroxycarboxylic diol) acid).

The compounds (2) that are particularly preferred in accordance with the invention are those chosen from the group comprising 2,2-di(hydroxymethyl)-acetic acid, 2,2-dihydroxymethylpropionic acid, 2,2-dihydroxymethylbutyric acid and 2,2-dihydroxymethylpentanoic acid.

The di- or polyisocyanate (3) may be chosen in particular from the group comprising hexamethylene diisocyanate, isophorone diisocyanate (IPDI), tolylene diisocyanate, diphenylmethane 4,4′-diisocyanate (DPMD) and dicyclohexylmethane 4,4′-diisocyanate (DCMD), methylenebis(p-phenyl) diisocyanate, methylenebis(4-cyclohexyl isocyanate), isophorone diisocyanate, toluene diisocyanate, 1,5-naphthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4- and 2,6-toluene diisocyanate, 2,2′-dichloro -4,4′-diisocyanatodiphenylmethane, 2,4-dibromo -1,5-diisocyanatonaphthalene, 1,4-diisocyanatobutane, hexane 1,6-diisocyanate and cyclohexane 1,4-diisocyanate.

The polycondensate may be formed using an additional compound (4) acting in general as chain extender for the polycondensate. These compounds (4) may be chosen from the group especially comprising saturated or unsaturated glycols such as ethylene glycol, diethylene glycol, neopentyl glycol and triethylene glycol, amino alcohols such as ethanolamine, propanolamine and butanolamine, heterocyclic, aromatic, cycloaliphatic and aliphatic primary amines, diamines, carboxylic acids such as aliphatic, aromatic and heterocyclic carboxylic acids, for instance oxalic acid, succinic acid, glutaric acid, adipic acid, sebacic acid and terephthalic acid, and aminocarboxylic acids. The preferred compounds (4) are the aliphatic diols.

The polycondensates in accordance with the invention may also be formed from additional compounds (5) containing a silicone skeleton, such as polysiloxanes, polyalkylsiloxanes or polyarylsiloxanes, especially polyethylsiloxanes, polymethylsiloxanes and polyphenylsiloxanes, optionally comprising hydrocarbonbased chains grafted onto the silicon atoms.

The polyurethane and/or polyurea blocks of the polymer which are advantageously used have a base repeating unit corresponding to general formula I′ below:

—X′—B —X′—CO —NH —R —NH —CO — (I′)

in which:

X′represents O and/or NH,

B is a divalent hydrocarbon-based radical, this radical being substituted or unsubstituted, and

R is a divalent radical chosen from alkylene radicals of aromatic, C₁ to C₂₀ aliphatic or C₁ to C₂₀ cycloaliphatic type, these radicals being substituted or unsubstituted.

Preferably, the radical B is a C₁ to C₃₀ radical bearing a group containing one or more carboxylic function(s) and/or one or more sulfonic function(s), said carboxylic and/or sulfonic functions being in free form or else partially or totally neutralized with a mineral or organic base.

The radical R is advantageously chosen from radicals corresponding to the following formulae:

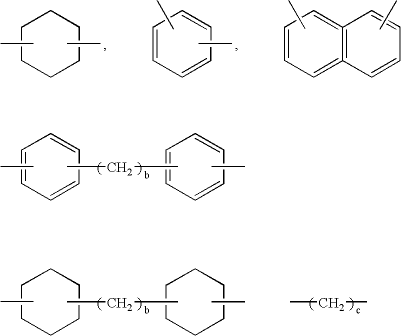

in which b is an integer between 0 and 3, and c is an integer between 1 and 20 and preferably between 2 and 12.

In particular, the radical R is chosen from hexamethylene, 4,4′-biphenylenemethane, 2,4′- and/or 2,6-tolylene, 1,5-naphthalene, p-phenylene, and methylene-4,4-bis(cyclohexyl) radicals and the divalent radical derived from isophorone.

The polycondensate used in accordance with the invention comprising at least one polyurethane and/or polyurea block may advantageously also comprise at least one polysiloxane block, the base repeating unit of which corresponds, for example, to the general formula (II′) below:

—X′—P —X′—CO —NH —R —NH —CO — (II′)

in which

P is a polysiloxane segment,

X′ represents O and/or NH, and

R is a divalent radical chosen from the alkylene radicals of aromatic, C₁ to C₂₀ aliphatic and C₁ to C₂₀ cycloaliphatic type, these radicals being substituted or unsubstituted.

Advantageously, the polysiloxane segment P corresponds to the general formula below:

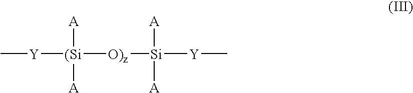

in which:

the radicals A, which may be identical or different, are chosen firstly from C₁ to C₂₀ monovalent hydrocarbon-based radicals that are free or substantially free of ethylenic unsaturation, and secondly from aromatic radicals,

Y represents a divalent hydrocarbon-based radical, and

z represents an integer chosen such that the average molecular weight of the polysiloxane segment is between 300 and 10 000.

In general, the divalent radical Y is chosen from the alkylene radicals of formula —(CH₂)ₐ—, in which a represents an integer that may be between 1 and 10.

The radicals A may be chosen from alkyl radicals, in particular methyl, ethyl, propyl, isopropyl, butyl, pentyl, hexyl, octyl, decyl, dodecyl and octadecyl radicals, cycloalkyl radicals, in particular the cyclohexyl radical, aryl radicals, especially phenyl and naphthyl, arylalkyl radicals, especially benzyl and phenylethyl, and also tolyl and xylyl radicals.

Preferably, the polycondensate(s) is (are) chosen from polycondensates comprising at least one silicone unit.

The distribution means, which forms part of the aerosol device, generally consists of a distribution valve controlled by a distribution head, itself comprising a nozzle via which the aerosol composition is vaporized.

In accordance with the invention, the initial flow rate of aerosol composition is measured for a temperature in the region of 20° C. inside the aerosol device. In practice, the aerosol device is placed at room temperature and the initial flow rate of aerosol composition is measured when thermodynamic equilibrium is reached.

The initial flow rate is the average amount of product leaving over 50 seconds from the aerosol device that has not been used previously. It is expressed in grams per second.

The initial flow rate of the aerosol composition (DA_{C}) corresponds to the amount of aerosol composition (fluid+propellant) exiting per unit of time from the aerosol device that has not been used previously. It is expressed in g/s and is measured by the difference between the weight of the aerosol before (M₀) and after (M₁) 10 seconds of vaporization:

D_{AC}=(M₀−M₁) /10.

Advantageously, the aerosol device according to the invention is suitable for obtaining an initial flow rate of aerosol composition of greater than or equal to 0.6 gram per second and less than or equal to 1.5 grams per second.

The initial flow rate of aerosol composition from the devices according to the invention depends firstly on the composition and secondly on the distribution means, the two needing to be suitable to obtain the desired characteristics.

The particular characteristics defined above may be obtained by selecting suitable distribution means and/or by modifying the formulation.

The polycondensates used in accordance with the invention may be soluble in the cosmetically acceptable medium or alternatively may form a dispersion in this medium. The dispersion may then comprise at least 0.05% of surfactant for dispersing the polycondensate and keeping it in dispersion.

According to the invention, any type of surfactant may be used in said dispersion, but preferably a nonionic surfactant. The mean particle size of the polycondensate in the dispersion is preferably between 0.1 and 1 micron.

The composition in accordance with the invention advantageously comprises, as a relative weight proportion relative to the total weight of the composition, between 4.8% and 15% of the polycondensate comprising at least one polyurethane and/or polyurea block, and even more advantageously between 5% and 12% of this polycondensate.

The composition in accordance with the invention advantageously comprises, as a relative weight proportion relative to the total weight of the composition, between 0.5% and 3% of the fixing polymer with a glass transition temperature of between 40 and 200° C., and even more advantageously between 0.7% and 2% of this fixing polymer.

The glass transition temperature of the fixing polymer (ii) is advantageously between 50° C. and 180° C. and even more preferentially between 60° C. and 170° C.

Among the fixing polymers with a glass transition temperature of between 40° C. and 200° C. that may be mentioned most particularly is the polyvinylcaprolactam sold by the company BASF, under the name Luviskol Plus.

In accordance with the invention, the organic solvent is chosen especially from the group comprising C₁ to C₄ alcohols such as ethanol or isopropanol, acetone, methyl ethyl ketone, methyl acetate, butyl acetate, ethyl acetate, dimethoxyethane and diethoxyethane, and mixtures thereof. Ethanol is preferably used.

The relative weight proportion, relative to the total weight of the composition, of propellent gas in the composition is advantageously between 15% and 85%, more advantageously between 25% and 60% and even more advantageously between 30% and 55%.

In accordance with the invention, the propellent gas preferably used is a gas that is soluble or insoluble in the composition, such as dimethyl ether, fluoro or nonfluoro hydrocarbons, common liquefied gases or a mixture of these propellent gases. Dimethyl ether is preferably used.

The compositions in accordance with the invention may moreover contain conventional cosmetic additives chosen especially from fatty substances, thickeners, softeners, antifoams, moisturizers, antiperspirants, basifying agents, colorants, pigments, fragrances, preserving agents, surfactants, hydrocarbon-based polymers other than those of the invention, volatile or nonvolatile silicones, especially anionic silicones, polyols, proteins and vitamins.

In particular, it may be advantageous to add to the composition other fixing polymers, such as nonionic, anionic, cationic or amphoteric fixing polymers, with a glass transition temperature of less than 40° C.

The invention may be understood more clearly with the aid of the nonlimiting example below.

### EXAMPLE

A device in accordance with the invention containing composition 1 below is produced.

| | |
|---|---|
| Luviset Si Pur sold by BASF | 5% |
| Luviskol Plus sold by BASF | 1% |
| Demineralized water | 14% |
| Ethanol | 35% |
| Dimethyl ether | 45% |

The initial flow rate measured at 20° C. is 0.58 gram per second.

This device is used firstly to apply composition 1 to wigs of European chestnut-brown hair, on which blow-drying was performed.

The blow-drying is particularly resistant to wind, the force of which is about 60 km/hour, modeled by a blower.

Blow-drying is performed on ten individuals. Composition 1 is applied to their hair. The hairstyle shows satisfactory hold for at least 4 hours. The individuals, hair is styled after 4 hours. The hair disentangles easily and has a pleasant feel.

1. An aerosol device comprising a container containing a hair composition formed from a fluid and at least one propellant, and also means for dispensing the composition, characterized in that:
(1) the composition comprises, in a cosmetically acceptable medium:
(i) between 4.5% and 20% by weight, relative′ to the total weight of the composition, of at least one polycondensate comprising at least one polyurethane and/or polyurea block;
(ii) between 0.1% and 5% by weight, relative to the total weight of the composition, of at least one fixing polymer other than the polycondensate(s) (i), with a glass transition temperature of between 40 and 200° C.;
(2) the device is suitable for supplying an initial flow rate of aerosol composition of greater than or equal to 0.55 gram per second and less than or equal to 2 grams per second.

2. The device as claimed in claim 1, characterized in that the initial flow rate of aerosol composition is greater than or equal to 0.6 gram per second and less than or equal to 1.5 grams per second.

3. The device as claimed in any one of the preceding claims, characterized in that the glass transition temperature of the fixing polymer (ii) is between 50 and 180° C.

4. The device as claimed in any one of the preceding claims, characterized in that the glass transition temperature of the fixing polymer (ii) is between 60 and 170° C.

5. The device as claimed in any one of the preceding claims, characterized in that the polycondensate is formed by an arrangement of blocks, this arrangement being obtained especially from:
(1) at least one compound containing two or more than two active hydrogen atoms per molecule;
(2) at least one diol or a mixture of diols containing acid radicals or salts thereof;
(3) at least one di- or polyisocyanate.

6. The device as claimed in claim 5, characterized in that the compounds (1) are chosen from the group comprising diols, diamines, polyesterols and polyetherols, or a mixture thereof.

7. The device as claimed in claim 5, characterized in that compound (2) is a 2,2-hydroxymethylcarboxylic acid.

8. The device as claimed in claim 5, characterized in that compound (3) is chosen from the group comprising hexamethylene diisocyanate, isophorone diisocyanate, tolylene diisocyanate, diphenylmethane 4,4′-diisocyanate, dicyclohexylmethane 4,4′-diisocyanate, methylenebis(p-phenyl) diisocyanate, methylenebis(4-cyclohexyl isocyanate), isophorone diisocyanate, toluene diisocyanate, 1,5-naphthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4- and 2,6-toluene diisocyanate, 2,2′-dichloro-4,4′-diisocyanatodiphenylmethane, 2,4-dibromo-1,5-diisocyanatonaphthalene, 1,4-diisocyanatobutane, hexane 1,6-diisocyanate and cyclohexane 1,4-diisocyanate.

9. The device as claimed in claim 5, characterized in that the polycondensate is formed from at least one additional compound with a silicone skeleton, chosen from the group comprising polysiloxanes, polyalkylsiloxanes or polyarylsiloxanes, especially polyethylsiloxanes, polymethylsiloxanes and polyphenylsiloxanes, optionally comprising hydrocarbon-based chains grafted onto the silicon atoms.

10. The device as claimed in any one of claims 1 to 4, characterized in that the polyurethane and/or polyurea blocks of the polymer have a base repeating unit corresponding to the general formula I′ below:
—X′—B —X′—CO —NH—R—NH—CO— (I′)
in which:
X′ represents O and/or NH,
B is a hydrocarbon-based radical, this radical being substituted or unsubstituted, and
R is a divalent radical chosen from alkylene radicals of aromatic, C₁ to C₂₀ aliphatic or C₁ to C₂₀ cycloaliphatic type, these radicals being substituted or unsubstituted.

11. The device as claimed in claim 10, characterized in that B is a C₁ to C₃₀ divalent hydrocarbon-based radical.

12. The device as claimed in claim 10 or 11, characterized in that the radical R is chosen from the group comprising hexamethylene, 4,4′-biphenylenemethane, 2,4- and/or 2,6-tolylene, 1,5-naphthylene, p-phenylene, and methylene-4,4-bis(cyclohexyl) radicals and the divalent radical derived from isophorone.

13. The device as claimed in any one of claims 1 to 4, characterized in that the polycondensate has a base repeating unit corresponding to formula (II′):
—X′—P—X′—CO—NH—R—NH—CO— (II′)
in which
P is a polysiloxane segment,
X′represents O and/or NH, and
R is a divalent radical chosen from the alkylene radicals of aromatic, C₁ to C₂₀ aliphatic and C₁ to C₂₀ cycloaliphatic type, these radicals being substituted or unsubstituted.

14. The device as claimed in any one of the preceding claims, characterized in that it comprises, as a relative weight proportion, relative to the total weight of the composition, between 4.8% and 15% of the polycondensate comprising at least one polyurethane and/or polyurea block, and even more advantageously between 5% and 12% of this polycondensate.

15. The device as claimed in any one of the preceding claims, characterized in that it comprises, as a relative weight proportion, relative to the total weight of the composition, between 0.5% and 3% of the fixing polymer with a glass transition temperature of between 40 and 200° C., and even more advantageously between 0.7% and 2%.

16. The device as claimed in any one of the preceding claims, characterized in that the propellent gas is present at a relative weight concentration of between 15% and 70%, preferably between 25% and 60% and more advantageously between 30% and 50%.

17. The device as claimed in any one of the preceding claims, characterized in that the composition also contains conventional cosmetic additives chosen from the group comprising fatty substances, thickeners, softeners, antifoams, moisturizers, antiperspirants, basifying agents, colorants, pigments, fragrances, preserving agents, surfactants, hydrocarbon-based polymers other than those of the invention, volatile or nonvolatile silicones, especially anionic silicones, polyols, proteins and vitamins.

18. The device as claimed in any one of the preceding claims, characterized in that the composition contains at least one additional fixing polymer chosen from the group comprising nonionic, cationic, anionic or amphoteric fixing polymers with a glass transition temperature of less than 40° C.

19. A haircare process for shaping or holding the hairstyle, characterized in that it comprises the use of a device in accordance with any one of claims 1 to 18.

20. The use of a device as claimed in any one of claims 1 to 18, for the manufacture of an aerosol lacquer or spray.
